(19) United States
(12) Patent Application Publication
(10) Pub. No.: US 2026/0124343 A1
(43) Pub. Date: May 7, 2026

(54) SOFT TISSUE AUGMENT AND METHODS OF MAKING AND USING SAME

(71) Applicant: Stryker Corporation, Kalamazoo, MI (US)

(72) Inventors: Kyle Craig Pilgeram, San Jose, CA (US); J. Brook Burley, Mountain View, CA (US); James Flom, Redwood City, CA (US); Jeremy Graul, Denver, CO (US)

(73) Assignee: Stryker Corporation, Kalamazoo, MI (US)

(21) Appl. No.: 19/377,892

(22) Filed: Nov. 3, 2025

Related U.S. Application Data
(60) Provisional application No. 63/715,301, filed on Nov. 1, 2024.

(51) Int. Cl. A61L 27/40; A61F 2/30; A61F 2/40

(52) U.S. Cl. CPC A61L 27/40; A61F 2/30749; A61F 2/30756; A61F 2/40; A61F 2002/30062; A61F 2002/30971; A61L 2430/24

(57) ABSTRACT

A soft tissue augment includes a first layer formed of a first material, the first material being a biological material, a synthetic material, or a composite material including both a biological material and a synthetic material, the first layer defining a first surface including a perimeter region surrounding a center region, and a second layer attached to the first surface of the first layer and formed of a second material, the second material being a biological material, a synthetic material, or a composite material including both a biological material and a synthetic material, the second material being different from the first material, the second layer extending from the perimeter region into the center region.

### CROSS-REFERENCE TO RELATED PATENT APPLICATIONS

The application claims the benefit of and priority to U.S. Provisional Patent Application No. 63/715,301, filed Nov. 1, 2024, which is incorporated by reference herein in its entirety.

### BACKGROUND

Joints of the human body may be susceptible to damage or injury due to excessive wear through intensive activity, a traumatic event, or through a long period of expected wear, among other reasons. One such joint susceptible to injury is the shoulder, where the soft tissue of the rotator cuff may tear. Surgeons have a variety of potential repairs of this soft tissue at their disposal, including the use of sutures and suture anchors to, in the case of cuff repair, reattach the soft tissue to the bone.

Another implant gaining popularity in soft tissue repair is the use of a patch or sheet of material to augment the repair. Specifically, this augment material is intended to be positioned against the soft tissue to provide healing benefits to the tissue. For example, these augments can promote tissue growth and/or can provide support or strength to the soft tissue.

There is much debate over the best type of augment material. Generally, some prefer biological or biodegradable materials as they commonly provide support and a scaffold for tissue in-growth but naturally degrade over time, leaving behind only natural tissue. However, such materials are typically fragile and can be difficult to manipulate and secure to the tissue. Others prefer non-biodegradable, typically polymeric, materials, in theory at least, as they are stronger, more user-friendly, and provide enduring support to the tissue. However, surgeons generally prefer to limit the amount of polymeric materials they implant in a patient, particularly when positioned within the articulating areas of a joint (such as in contact with the rotator cuff tissue). As such, a need exists for an improved augment material that can provide the benefits desired by surgeons but limit the negative features of currently-used materials.

### SUMMARY OF THE INVENTION

In one aspect, the present application relates to a new soft tissue augment material with improved strength and usability while also providing the benefits of biological materials. In some instances, the augment may include at least one biological or degradable synthetic material and at least one non-biodegradable material.

In one aspect, the present disclosure is a soft tissue augment including a first material and at least one second material positioned along at least a portion of an outer perimeter of the first material, wherein the second material is attached to the first material along at least a portion of both the first and second materials.

The first material may be a biological material, a synthetic (degradable or non-degradable) material, or a composite material including both biological and synthetic components. The second material also may be a biological material, a synthetic (degradable or non-degradable) material, or a composite material including both biological and synthetic components.

In another aspect, the present disclosure is a method of making a soft tissue augment, including obtaining a first component formed of a first material as described above, obtaining a second component formed of a second material as described above, and attaching the first component to the second component along at least a portion of both the first and second components.

The attaching of the first component to the second component may include the use of sewing, felting, adhering, or stitching. The attaching of the first component to the second component may occur along at least a portion of an overlapping surface area of both the first and second components.

In a further aspect, the present disclosure is a method of making a soft tissue augment, including obtaining a first component formed of a first material as described above, obtaining a second component formed of a second material as described above, attaching the first component to the second component along at least a portion of both the first and second components to form the soft tissue augment, and implanting the soft tissue augment against a portion of soft tissue.

Implanting the augment may include the use of sewing, felting, adhering, or stitching of the soft tissue augment to the portion of soft tissue. The implanting may include positioning at least a portion of the second component against the soft tissue. The implanting may include positioning at least a portion of both the first component and the second components directly against the soft tissue. The implanting may include attaching the soft tissue augment to the soft tissue along at least one location within a surface area of the second component.

A further aspect of the present disclosure relates to a soft tissue augment including a first layer formed of a first material, the first material being a biological material, a synthetic material, or a composite material including both a biological material and a synthetic material, the first layer defining a first surface including a perimeter region surrounding a center region, and a second layer attached to the first surface of the first layer and formed of a second material, the second material being a biological material, a synthetic material, or a composite material including both a biological material and a synthetic material, the second material being different from the first material, the second layer extending from the perimeter region into the center region.

A further aspect of the present disclosure relates to a method of forming a soft tissue augment. The method includes providing a first layer formed of a first material, the first material including a biological material, a synthetic material, or a composite material including both a biological material and a synthetic material, the first layer defining a first surface including a perimeter region surrounding a center region, and coupling a second layer to the first surface of the first layer with the second layer extending from the perimeter region into the center region, the second layer formed of a second material, the second material being a biological material, a synthetic material, or a composite material including both a biological material and a synthetic material, the second material being different from the first material.

A further aspect of the present disclosure relates to a method including positioning an augment over soft tissue, the augment including a first layer formed of a first material, the first material being a biological material, a synthetic material, or a composite material including both a biological material and a synthetic material, the first layer defining a first surface including a perimeter region surrounding a center region, and a second layer attached to the first surface of the first layer and formed of a second material, the second material being a biological material, a synthetic material, or a composite material including both a biological material and a synthetic material, the second material being different from the first material, the second layer extending from the perimeter region into the center region, and fixating the augment to the soft tissue, wherein a fixation element engages with the second layer to secure the augment to the soft tissue.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1A illustrates a top view of an example of a first material which can become a portion of a soft tissue augment.

FIGS. 1B and 1C illustrate top views of various examples of a soft tissue augment consistent with the present disclosure.

FIG. 1D illustrates a section view of the soft tissue augment of FIG. 1B.

FIG. 1E illustrates a section view of the soft tissue augment of FIG. 1B according to an alternative embodiment.

FIGS. 1F and 1G illustrate top views of various examples of a soft tissue augment consistent with the present disclosure.

FIG. 1H illustrates a front view of the soft tissue augment of FIG. 1G.

FIGS. 2A-3 illustrate top views of various examples of a soft tissue augment consistent with the present disclosure.

FIGS. 4A-4D illustrate top views of various examples of a soft tissue augment including features for engagement with an augment spreader and delivery device.

FIG. 4E illustrates a section view of the soft tissue augment and the augment spreader and delivery device of FIG. 4B.

FIGS. 5A-5E illustrate top views of various examples of a soft tissue augment consistent with the present disclosure, including for use in applications such as augmentation of rotator cuff soft tissue.

FIG. 5F illustrates an example of an augment spreader and delivery device.

FIGS. 6A-6B illustrate top views of various examples of a soft tissue augment consistent with the present disclosure.

FIGS. 7A-7B illustrate top views of various examples of a soft tissue augment consistent with the present disclosure.

FIG. 8A illustrates a top view of an example of a soft tissue augment consistent with the present disclosure.

FIG. 8B illustrates a front view of the soft tissue augment of FIG. 8A.

FIG. 8C illustrates a front view of an example of a soft tissue augment consistent with the present disclosure.

FIG. 9A illustrates a top view of an example of a soft tissue augment consistent with the present disclosure.

FIG. 9B illustrates a front view of the soft tissue augment of FIG. 9A.

FIG. 9C illustrates a side view of the soft tissue augment of FIG. 9A.

FIG. 10A illustrates a top view of an example of a soft tissue augment consistent with the present disclosure.

FIG. 10B illustrates a section view of the soft tissue augment of FIG. 10A.

FIGS. 10C-10D illustrate top views of various examples of a soft tissue augment consistent with the present disclosure.

FIG. 11A illustrates a top view of an example of a soft tissue augment consistent with the present disclosure.

FIG. 11B illustrates a front view of the soft tissue augment of FIG. 11A.

FIGS. 12A-12D illustrate a method of forming the soft tissue augment of FIG. 11A consistent with the present disclosure.

FIGS. 13A-13C illustrate a method of forming the soft tissue augment of FIG. 11A consistent with the present disclosure.

FIG. 14A illustrates a top view of an example of a soft tissue augment consistent with the present disclosure.

FIG. 14B illustrates a section view of a portion of the soft tissue augment of FIG. 14A.

FIGS. 15A-15D illustrate a method of forming the soft tissue augment of FIG. 14A consistent with the present disclosure.

FIG. 16 illustrates a method of fixating an augment consistent with the present disclosure.

### DETAILED DESCRIPTION

As used herein, the term “distal” refers to that portion of an instrument or component thereof which is farther from the user while the term “proximal” refers to that portion of the instrument or component thereof which is closer to the user. The term “medial” means closer to or toward the midline of the body, and the term “lateral” means further from or away from the midline of the body. The term “inferior” means closer to or toward the feet, and the term “superior” means closer to or toward the crown of the head. As used herein, the terms “about,” “approximately,” “generally,” and “substantially” are intended to mean that slight deviations from absolute are included within the scope of the term so modified.

The soft tissue augments discussed herein may be used in a variety of applications. While use in the repair of rotator cuff is the primary example used throughout, the augments may be used to supplement or support other soft tissues, such as the Achilles, biceps, soft tissue in the knee, hip, elbow, wrist, hand/foot, etc.

Soft tissue augment 10 can be formed of any material or combination of materials desired. For example, synthetic materials such as polymeric materials can form at least part of augment 10. Examples include polypropylene, polytetrafluoroethylene, polyester, or combinations thereof, and such materials may be woven or nonwoven. As such, one such specific example may be nonwoven polyester. Polyester is a synthetic, non-biodegradable material, and has various benefits including good strength, even in a thin dimension, good fixation to tissue, and is biocompatible in that it does not irritate surrounding tissue. Such materials like polyester can also serve as a scaffold for new tissue ingrowth. However, augment 10 formed entirely of polyester, or another synthetic non-biodegradable material, can cause issues in the event of, for example, infection, as the entire augment may need to then be removed which can be traumatic to the patient.

In order to account for the benefits of these synthetic materials while lessening the drawbacks and risks associated with them, in one aspect of a soft tissue augment of the present disclosure, the augment is formed of a combination of at least one first material and one second material. The “first material” may be a biological material, a synthetic material (which may be degradable or non-degradable), or a composite material including both biological and synthetic components. The “second material” also may be a biological material, a synthetic material (which may be degradable or non-degradable), or a composite material including both biological and synthetic components.

Examples of the first material and the second material include, but are not limited to, biological materials including collagen (including cross-linked collagen, non-cross-linked collagen, reconstituted collagen, native collagen (e.g. dermal tissue), human collagen, bovine collagen, and/or xenograph collagen), polysaccharides, polyurethane, poly(urethane urea), and polypeptides, degradable synthetic materials including polylactic acid (PLA), polycaprolactone (PCL), polyglycolide (PGA), poly(lactic-co-glycolic acid) (PLGA), poly(l-lactide-co-ε-caprolactone) (PLCL), poly-L-lactic acid (PLLA), polyhydroxyalkanoates (PHAs), poly D-lactide (PDLA), poly DL-lactic acid (PDLLA), polycaprolactone (PCL), polyanhydrides, and polydioxanone (PDO), and non-degradable synthetic materials including, but not limited to, polypropylene, polytetrafluoroethylene, polyester, and polyethylene. In various embodiments, the second material may include, but not be limited to, any of the above materials, and may be different than the first material or may be the same material as the first material. In many embodiments, the second material may have a greater strength than the first material. As used herein, the term “greater strength” may be defined to include one or more of greater tensile strength, greater tear strength and/or greater puncture resistance.

In various examples disclosed herein, one or more portions of a second material may be bonded, stitched, or otherwise coupled to a patch or sheet of a first material to reinforce and/or stiffen the patch or sheet. In some embodiments, electrospinning or other material deposition processes may be used to deposit and bond the second material onto the first material. It should be noted that in various embodiments, the first material may be referred to as a first layer formed from the first material, and the second material may be referred to as a second layer formed from the second material. The second layer may include various portions as discussed below. Further, in some embodiments, the second layer may include portions formed from different materials.

Soft tissue augments may be coupled to native tissue at an implantation site using various methods, including extending fixation elements (e.g., staples, tacks, filaments, sutures, etc.) through the augment and into the native tissue. In the case where sutures or other filaments are used, the augment may be stitched to the native tissue or passed through anchors, which are secured to the native tissue. One or more sutures may also be used to couple the soft tissue augment to one or more suture anchors. Coupling the second material to the first material may reinforce the augment in the locations that the fixation elements pass through. For example, suture, filaments, tacks, staples, etc. may be pushed through the second material instead of or in addition to being pushed through the first material. Passing the fixation elements through the second material may help to resist tearing of the first material and may provide a more secure attachment of the augment to the native tissue.

In some embodiments, as discussed above, the first material may be collagen, which is a natural tissue that can serve as a scaffold for tissue ingrowth and which is slowly replaced or modified by natural tissue. Over time, in embodiments in which the second material is not degradable, the augment may change from a starting construction (e.g., at the time of implantation) including the first material and the second material, to an end structure (e.g., at a later point in time post-implantation) including only the second material (e.g., polyester) with the other material(s) having been degraded, substantially degraded or otherwise modified to the point that this first material has largely, if not entirely, been replaced by natural tissue. As such, the augment in this aspect results in an implant that supports the repaired tissue and provides a scaffold for new tissue ingrowth, while resulting in a much smaller amount of permanent, non-degradable material left in the patient over the long term.

As discussed in further detail below, in some embodiments, the second material is used to provide sufficient strength, stiffness, reinforcement and/or delivery compatibility to the patch or sheet of first material. In some embodiments, the second material may be configured as a fibrous network of material, such as non-woven polyester (e.g., polyethylene terephthalate). A specialized instrument may be used to push fibers of the second material through the patch or sheet of first material, coupling the two layers together. In some embodiments, the fibers of the second material may be additionally or alternatively pushed into the native tissue at the implantation site to secure the augment to the tissue.

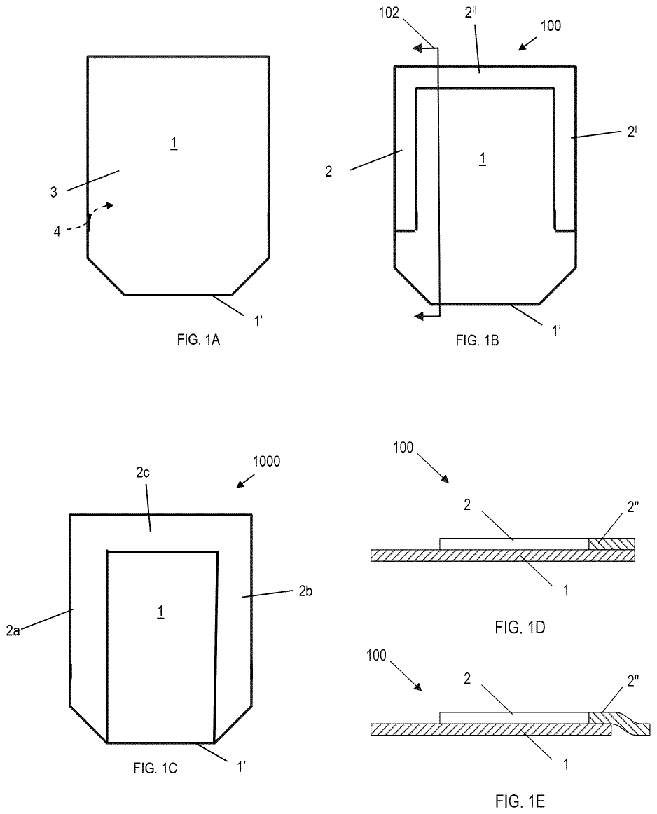

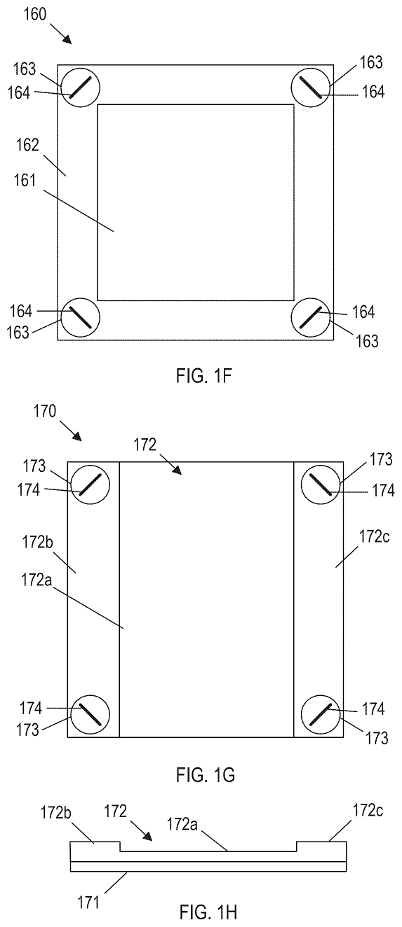

As illustrated in the various figures accompanying this disclosure, in some aspects, it is desirable to minimize the amount of the second material (e.g., polyester). For instance, using FIG. 1B as an example, the second material may be positioned within the augment 10 construct in particular locations where it will provide benefit, such as along the corners and/or edges of the first material to provide areas of added strength and structure where augment 10 can be attached to soft tissue. Positioning the second material at the corners of the augment 10 may be useful as the corners are typically attached to the soft tissue to ensure the augment 10 generally remains flat with as much of its surface area against the soft tissue surface as possible. That said, the second material may be positioned at any location and along any portion of the augment 10 as desired or useful for a particular application, as will be discussed in greater detail herein. In other embodiments, the second material may extend over the entire surface area of the first material to provide full coverage of one side of the sheet or patch of first material. This may allow for reinforced stitching or fixation of the augment 10 at any point across the augment 10. In some embodiments, a relatively thin layer of a second material may extend over the entire surface area of the first material, and either a third material or a thicker layer of the second material may be provided across less than the entire surface area of the first material (e.g., to reinforce particular portions, as discussed above). For example, in some embodiments, the second material may extend completely over at least a first surface of the first material, but the thickness of the second material may be deliberately varied in order to provide a thicker layer of the second material in areas where additional strength is desired and a thinner layer of the second material in areas where less strength is desired. According to other embodiments, the second material may extend over less than a full surface of the first material but may still have varying thicknesses.

FIG. 1A provides an example of a first material 1 which is one component of a soft tissue augment of the present disclosure. While first material 1 is illustrated as generally hexagonal, first material 1 may have any shape desired such as any polygon, rectangle, square, circle, oval, annulus, triangle, or the like. As referred to throughout the present disclosure, the first material 1 may have a top surface 3 (i.e., facing out of the page) and a bottom surface 4 (i.e., facing into the page). FIG. 1B illustrates one example of a second material 2, 2′, 2″ positioned onto the first material 1 to form soft tissue augment 100. As illustrated, the second material may be a monolithic shape positioned along at least a portion of the surface area of first material 1. Second material includes portions 2, 2′, 2″ that overlie different portions of first material 1. In another example, the second material may only include portions 2, 2″, forming an L-shape. Still further, second material may include only portions 2, 2′, thereby forming two separate portions of second material overlying different portions of first material 1. Similarly, portions 2, 2′, 2″ may all be separate portions that are each applied to first material 1 to form an overall pattern, shape, and size of the second material relative to the first material 1. FIG. 1D illustrates a section view of augment 100 along plane 102. As shown in FIG. 1D, the portions 2, 2″ of the second material of equal thickness and are positioned on and extend from a surface of the first material 1. In some embodiments, as described herein, different portions of the second material may have different thicknesses. As illustrated in FIG. 1D, the second material 2, 2′, 2″ may overlie the first material 1 such that the outer edges of second material 2, 2′, 2″ generally align with the outer edges of the first material 1. However, second material 2, 2′, 2″ may be positioned along any portion of the first material 1. FIG. 1E illustrates a section view of augment 100 along plane 102 according to an alternative embodiment. As shown in FIG. 1E, the second material portion 2″ is positioned to only partially overlie the first material 1 and at least partially extend beyond the outer edge of the first material 1. This may allow, for example, fixation elements (e.g., tacks, staples, sutures, filaments, etc.) to pass through the second material portion 2″ without passing through the first material 1. It should be understood that any portion of the second material in the embodiment disclosed herein may extend beyond an outer perimeter of the first layer. In other embodiments, at least a portion of the second material 2, 2′, 2″ may be positioned to overlie a portion of the first material 1, extend beyond the edge of the first material 1, and then be folded underneath the first material 1. This example may form a wrap-around structure of the second material 2, 2′, 2″ around an edge of the first material 1.

FIG. 1C illustrates another example of a second material 2a, 2b, 2c positioned onto the first material 1 to form soft tissue augment 1000. As illustrated, the second material may be a monolithic shape positioned along at least a portion of the surface area of first material 1. Variations on the positioning, shape, and size of second material 2a, 2b, 2c may be similar to that set forth above as to second material 2, 2′, 2″ of FIG. 1B. Different from FIG. 1B, however, is that second portions 2a, 2b extend towards lateral edge 1′ of first material 1, thereby extending along the entire side edges of first material 1 to lateral edge 1′. Further, above as to FIG. 1B, portions 2a, 2b, 2c may all be a single, monolithic portion that is applied as a single piece to the first material 1, or may be separate portions that are each applied to first material 1 to form an overall pattern, shape and size of the second material relative to the first material 1.

As with any of the examples set forth herein, the first material 1 of FIGS. 1A-1C may be one of the aforementioned first materials, while, as to FIGS. 1B and 1C, the second material portions 2, 2′, 2″, 2a, 2b, 2c may be one of the aforementioned second materials. The portions 2, 2′, 2″, 2a, 2b, 2c are illustrated here as forming a border along three sides of augment 100, 1000, respectively, but the shape, design, number of such portions can be any desired. As to the illustrated design in FIGS. 1B and 1C, augment 100, 1000 may be particularly useful for common repairs of the rotator cuff. Typically, the augment 100, 1000 is positioned above or below the cuff tissue, where portion 2″, 2c is positioned in a medial position while the bare edge of the first material 1′ is positioned in a lateral position. The augment 100, 1000 may be coupled to native tissue at the implantation site, by, for example, extending fixation elements (e.g., staples, tacks, filaments, sutures, etc.) through the second material portions 2, 2′, 2″, 2a, 2b, 2c. The second material portions 2, 2′, 2″, 2a, 2b, 2c may reinforce the connection of the augment 100, 1000 to the native tissue relative to a fixation element passed only though the first material 1. The medial position of portion 2″, 2c is likely to be affixed to soft tissue present in that position, and thus portion 2″, 2c will provide strength to the fixation location. In some embodiments, the portions 2, 2′, 2a, 2b may be affixed to native tissue at the implantation site either in addition to 2″ and 2c or instead of 2′ and 2c, including along the lateral portion opposite the medial portion. In some embodiments, the second material portions 2, 2′, 2a, 2b as shown in FIGS. 1B and 1C, may not be present along the lateral edge of the first material 1.

FIG. 1F illustrates an augment 160 according to an alternative embodiment. The augment 160 includes a first material 161 and a second material 162 that extends around the entire perimeter of the first material 161. Augment 160 illustrates exemplary fixation locations 163 through which fixation elements, shown as staples 164, may be pressed through at least the second material 162 to couple the augment 160 to native tissue at an implantation site. As discussed above with respect to FIGS. 1D and 1E, the second material 162 may be aligned with the edge of the first material 161, in which case the staple 164 may extend through both the first material 161 and the second material 162 or may extend beyond the edge of the first material 161, in which case the staples 164 may extend through only the second material 162. It should be understood that the fixation locations 163 are examples, and that fixation elements may pass through augments at various locations. However, passing fixation elements through the second material or through both the first material and the second material may provide a more secure attachment of the augment to the native tissue at the implantation site than passing fixation elements through the first material alone, due to, for example, the material properties and thickness of the second material. As discussed in further detail below with respect to FIGS. 1G and 1H, in some embodiments, the second material may include portions having different thicknesses. For similar reasons, passing fixation elements through thicker portions of the second material may provide a more secure attachment of the augment to the native tissue. In some embodiments, fixation elements may be positioned generally around a perimeter portion of the augment, which may minimize interruptions of the first material in the region covering the surgical site.

As illustrated in at least FIGS. 1B, 1C, and 1F, in various embodiments, the second material may be positioned around the perimeter of the first material to define linear strips of second material. The second material may define linear strips on one side, two sides, three sides, or all four sides of the perimeter of a substantially rectangular first portion. According to other embodiments, the second material may be positioned around all or a portion of the perimeter of the first material while being in a shape other than linear strips. Regardless of the shape of the first material, the second material may define linear strips around at least 25% of the perimeter of the first material, at least 50% of the perimeter of the first material, or at least 75% of the perimeter of the first material. It should be understood that the “linear strips” of second material may be separate pieces of material or may be portions of a single piece of material. For example, the second material 162 in FIG. 1F may be a single piece of material but defines four linear strips around the perimeter of the first material 161. The linear strips may not necessarily be rectangular or otherwise include parallel sides, but may allow a line to pass therethrough.

Referring now to FIGS. 1G and 1H, a top view and a front view of an augment 170 are shown, respectively, according to an alternative embodiment. The augment 170 includes a first material 171 and a second material 172 that covers an entire surface of the first material 171. The second material includes two edge portions 172b, 172c that extend along edges of a first surface of the first material 171 and a center portion 172a that covers the rest of the first surface between the two edge portions 172b, 172c. The edge portions 172b, 172c are thicker than the center portion 172a. as discussed above, it may be beneficial for fixation elements to be passed through a thicker portion of material. Thus, augment 170 illustrates example fixation locations 173 through which fixation elements, shown as staples 174, may be pressed through at least the thicker edge portions 172b, 172c of the second material to couple the augment 170 to native tissue at an implantation site. In various embodiments, the three portions 172a, 172b, 172c of second material may be formed of a single piece of material. In other embodiments, the center portion 172a may cover the entire surface of the first material 171, and the two edge portions 172b, 172c may be applied on or coupled to the top of the center portion 172a. In still other embodiments, each portion 172a, 172b, 172c may be a separate component applied on or coupled to the surface of the first material 171.

As noted above, while the second material is designated as portions 2, 2′, 2″, 2a, 2b, 2c in FIGS. 1B-1C, these portions may be portions of a monolithic structure of the second material, or alternatively could be individual, separate portions each attached to the first material 1. The exact configuration of the portion(s) of second material can be any as desired for the most beneficial manufacturing, supply and use applications. Further, the portions 2, 2′, 2″, 2a, 2b, 2c, like the first material 1, could be of any shape or size desired. For instance, instead of the three polygonal-shaped portions 2, 2′, 2″, 2a, 2b, 2c illustrated in FIGS. 1B-1C, the portions, such as when considered individually as portions 2, 2′, 2″, 2a, 2b, 2c, could be polygons, circles, triangles, squares, annular, or could take on a different shape or size altogether (both individually and in combination with other portions of second material).

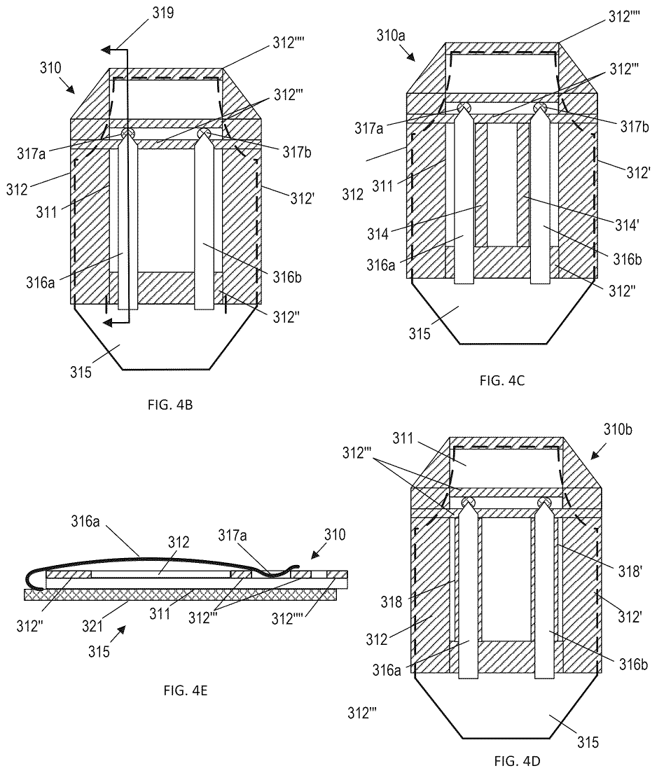

Still further, fewer portions, or portions of smaller size, could also be applied to the first material 1, or alternatively, additional portions of second material can be added to the first material 1 if desired. In one such example, the lateral end 1′ of the first material 1 that is illustrated as free of any second material, could instead have a portion of the second material along that edge, and/or at least part of the top and/or bottom surfaces 3, 4 of first material 1, away from the outer edges of the first material 1, could include at least some second material (e.g., see portions 314, 314′ in FIG. 4C).

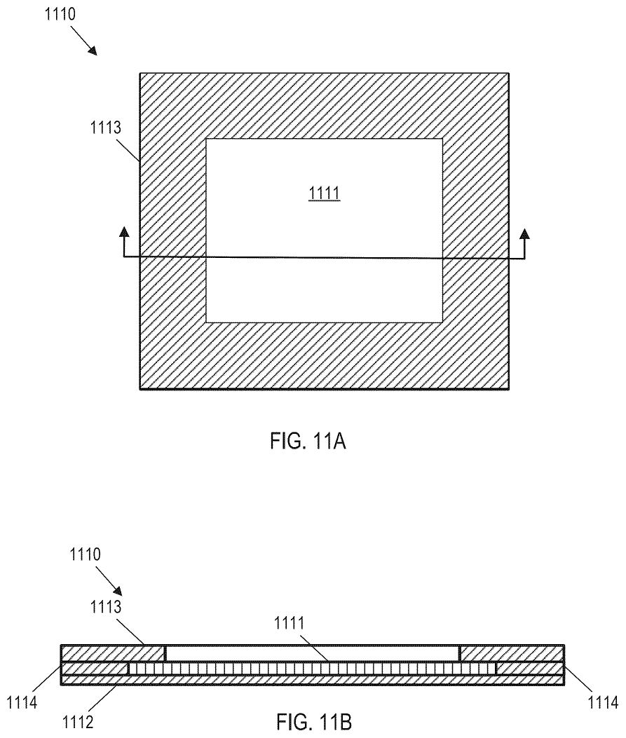

Similarly, in other aspects, the second material portions 2, 2′, 2″, 2a, 2b, 2c could be positioned on the top surface 3, as illustrated in FIGS. 1B-1C, and/or the bottom surface 4 of first material 1. As discussed further below, it may be beneficial to have the second material positioned in between the first material and the soft tissue being repaired, such that the second material forms a secure attachment to both the first material and the soft tissue. This may be particularly true in the exemplary method of making and using with the sewing or felting process, where, for example, nonwoven polyester (as the second material) is sewn directly into both the first material (prior to implantation) and into the soft tissue (during implantation). That said, different applications may warrant other configurations where, for instance, it is beneficial to position the first material in between the second material and the soft tissue. Still further, certain applications may benefit from positioning second material in between two separate layers of first material, or in a pocket in between connected layers of first material. In yet another example, portions of second material may be positioned on both the top and bottom surfaces of the first material, and such portions of second material on the top surface of the first material may or may not overlap portions of second material on the bottom surface of the first material. Still further, as noted above, portions of second material could wrap around edge(s) of the first material such that a portion extends along a portion of both the top and bottom surfaces. These embodiments are discussed in further detail below with respect to FIGS. 11A-13C. Such various configurations may provide additional utility of the soft tissue augment for various applications where, for example, sewing or felting (or stitching, adhering, etc.) may be performed on both sides of the augment or may attach to two different soft tissue locations, or overlapping portions of second material may provide additional strength and support to the first material, or the like.

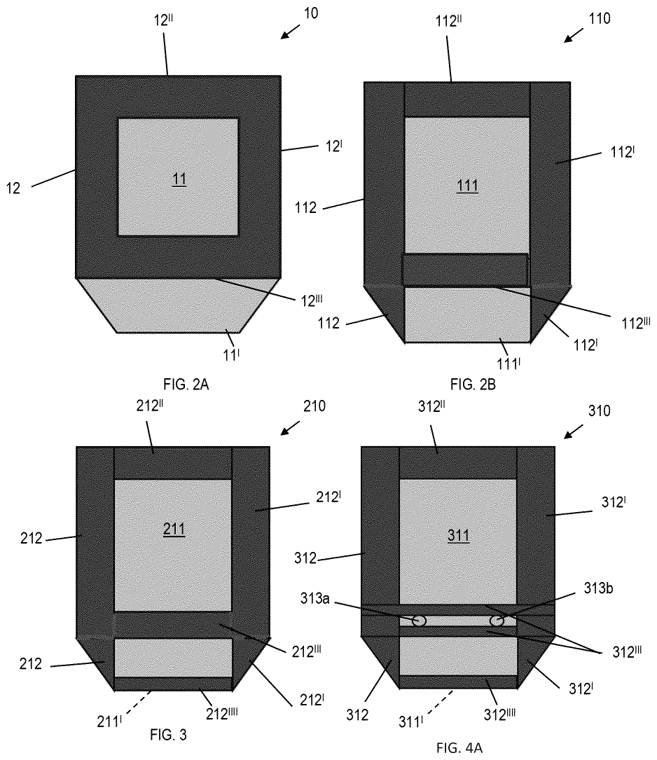

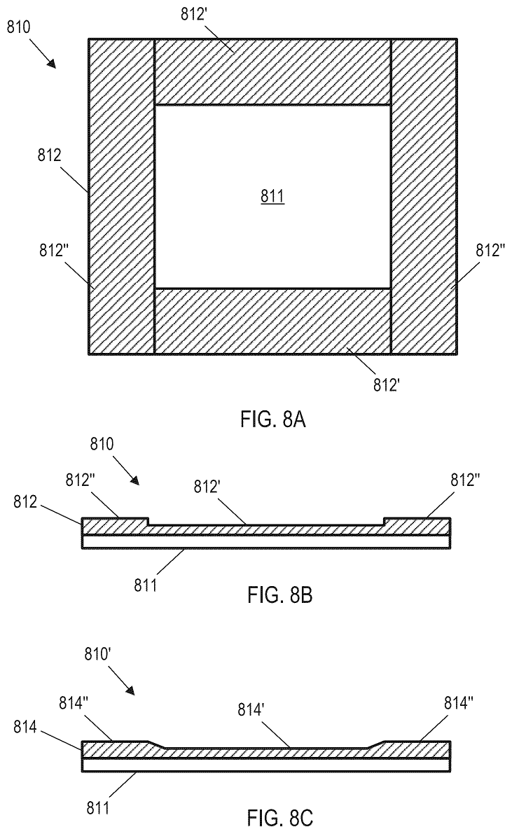

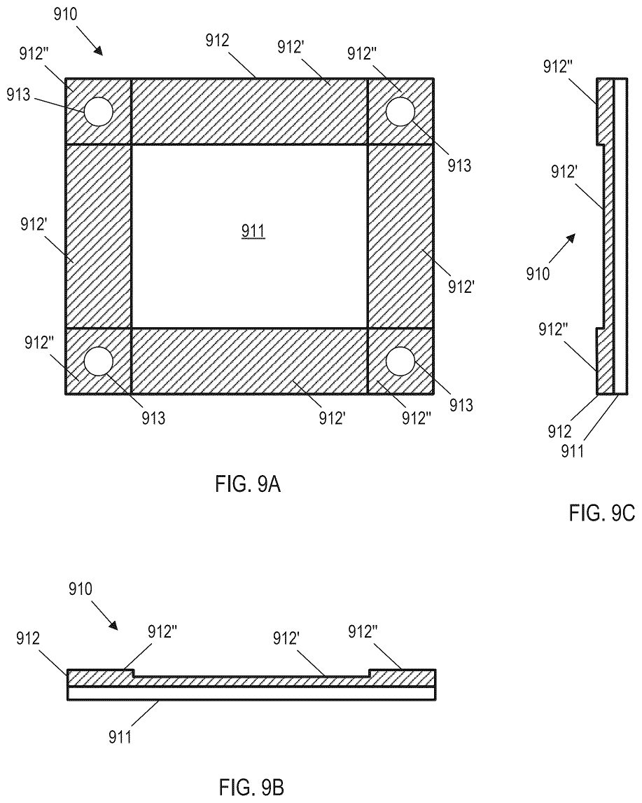

FIG. 2A provides yet another example of the structure of a soft tissue augment 10. While in the examples provided in FIGS. 1B and 1C, the second material portions 2, 2′, 2″, 2a, 2b, 2c are coupled only to a perimeter region of the first material, in some embodiments, second material portions may extend from the perimeter region into or across a center region surrounded by the perimeter region. The portions extending across the center region may be specifically designed to account for the particular application for the augment. For example, in some embodiments, at least some of the second material portions may be shaped to interface with an augment spreader and delivery device or may be shaped to provide structure to the augment without interfering with sutures in a double row rotator cuff repair. In some embodiments, at least some of the second material portions may be shaped to provide additional strength and/or abrasion resistance in areas with sutures, anchors, and/or other fixation elements. In the example shown in FIG. 2A, augment 10 includes a first material 11 and a second material, illustrated as portions 12, 12′, 12″, 12″. Portions 12, 12′, 12′, 12′″ may overlap first material 11 such that their outer edges are substantially aligned with outer edges of first material 11, as discussed above as to FIG. 1B. Alternatively, portions 12, 12′, 12′, 12′″ can extend further out from the boundary of the first material 11, or can even be positioned along any other locations of first material, such as more towards the interior surface and away from the edge(s) of the first material 11. Portion 12′″ may be optional, as are any of the portions of the second material. Further, as is the case with any of the examples in the present disclosure, any one of the portions 12, 12′, 12″, 12′″ could be of different dimensions from one another. For instance, portion 12′″ could be slightly thicker than the other portions 12, 12′, 12″ so as to provide a structure along the top surface of the first material 11 that is more pronounced (i.e., extends to be taller than the other portions 12, 12′, 12″ off of the top surface of the first material 11), which could be useful in handling, such as for engaging an augment spreader and delivery device or for other purposes, such as for providing additional support to the augment 10, as described in greater detail below. Embodiments in which portions of the second material vary in thickness are discussed in further detail below with respect to FIGS. 8A-9C. As noted above, first material 11 is also shown as having a hexagonal shape with a tapering lateral end, though this is optional and the shape of first material 11 may be any desired.

Furthermore, as discussed above as to FIGS. 1B-1C, where augment 10 is intended for use in repairing the rotator cuff soft tissue, the lateral edge 11′ of first material 11 may remain free of any second material, though optional portion 12′″ could provide utility in that, at that position on first material 11 (i.e., more medial from the lateral edge), soft tissue may be present to which the augment 10 could be attached.

FIG. 2B illustrates another aspect of a soft tissue augment, such as augment 110 which is similar to augment 10 such that like numerals denote like structures, and which includes a first material 111 and a second material formed of portions 112, 112′, 112″. In this instance, portions 112, 112′ are similar to portions 12, 12′ of FIG. 2A except portions 112, 112′ have a right trapezoidal shape (similar to portions 2a, 2b in FIG. 1C) which extends further along the edges to the tapered lateral edge 111′ of the first material 111. The additional length of the second material may be useful in some applications where a user desires to, for example, attach all four corners to tissue. The additional length of portions 112, 112′ may also serve to further strengthen and support the lateral edges of the first material 111, which may be weaker than the second material portions 112, 112′. The optional additional portion 112′″ is also illustrated in FIG. 2B, though as above as to FIG. 2A, this portion is optional and is simply positioned along a portion of the surface area of first material 111. As in the other embodiments, any combination or all of second portions 112, 112′, 112″, 112′″ may be monolithic or be one or more separate portions, and indeed, any individual designated portions 112, 112′, 112″, 112′″ may themselves be multiple portions that are brought together on first material 111 to form the desired pattern. To that end, it should be understood that any lines of demarcation that may be observed within the surface area of the second portions 112, 112′, 112″, 112′″, or in between one portion and another portion, should not be viewed as limiting, but rather any such lines are simply artifacts from the drafting of the figures. As noted throughout this application, each of the second portions may be any combination of size and shape relative to the other portions, or may together be a complete monolithic structure for application to the first material.

FIG. 3 illustrates yet another aspect of a soft tissue augment, such as augment 210 which is similar to augment 10, 110 such that like numerals denote like structures, and which includes a first material 211 and a second material formed of portions 212, 212′, 212″. In this instance, portions 212, 212′ are similar to portions 112, 112′ of FIG. 2B, and a further portion 212′″ is optionally present, similar to FIG. 2B. Still further, another portion 212″″ may be positioned on first material 211, such as along the lateral edge 211′ of first material 211 which may provide additional strength to the augment 210, particularly along the lateral edge 211′ of the first material 211. Portion 212″″ also illustrates how the various portions can have differing dimensions and shapes from the other portions. For instance, portion 212″″ has a thinner width than the other portions 212, 212′, 212′, 212′″, which may provide the benefits of the second material to the lateral edge 211′ while still limiting the amount of the second material at that location. As in the other embodiments, any combination or all of second portions 212, 212′, 212″, 212′″, 212″″ may be monolithic or be one or more separate portions, and indeed, any individual designated portions 212, 212″, 212″, 212′″, 212″″ may themselves be multiple portions that are brought together on first material 211 to form the desired pattern. To that end, it should be understood that any lines of demarcation that may be observed within the surface area of the second portions 212, 212′, 212″, 212′″, 212″″, or in between one portion and another portion, should not be viewed as limiting, but rather any such lines are simply artifacts from the drafting of the figures. As noted throughout this application, each of the second portions may be any combination of size and shape relative to the other portions, or may together be a complete monolithic structure for application to the first material.

FIGS. 4A, 4B, and 4E illustrate another aspect of a soft tissue augment, such as augment 310 which is similar to augment 10, 110, 210 such that like numerals denote like structures, and which includes a first material 311 and a second material designated by portions 312, 312′, 312″. In this instance, portions 312, 312′ are similar to portions 112, 112′ of FIG. 2B, and a further portion 312′″ is present. Still further, another portion 312″″, similar to portion 212″″ of FIG. 3, may be positioned along the lateral edge 311′ of first material 311.

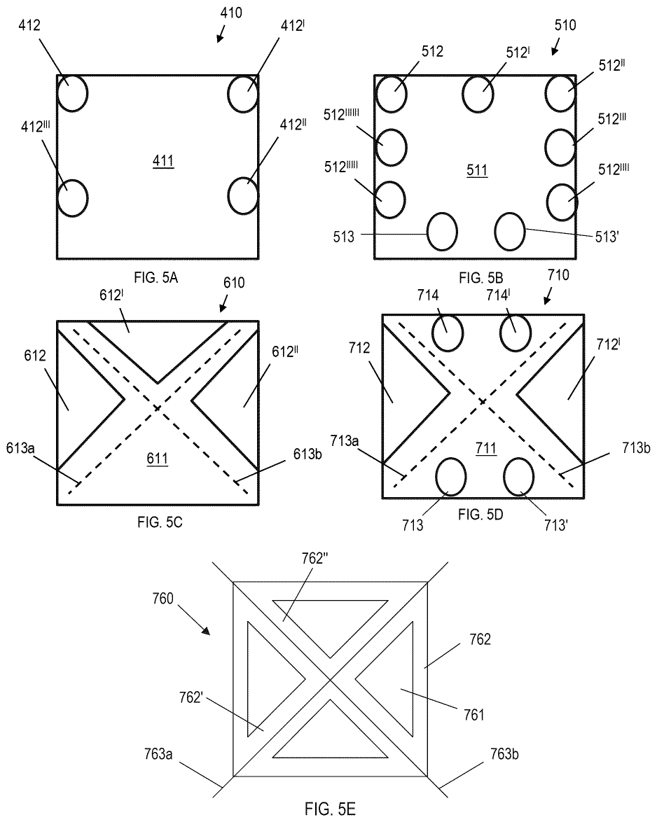

In this example, portion 312′″ may be positioned adjacent to and/or define at least one instrument engagement location 313a, shown as two locations 313a and 313b. As illustrated, portion 312′″ includes two portions that are spaced apart from one another. First material 311 may be exposed in between portions 312′″ to provide locations 313a, 313b in between portions 312′″ for engagement with an instrument. For example, as illustrated in FIGS. 4B and 4E, such an instrument may be an augment spreader and delivery device 315 which has attachment members 316a, 316b (e.g., clips, springs, etc.) that can engage locations 313a, 313b. FIG. 5F is reproduced from U.S. application Ser. No. 18/471,655, filed Sep. 21, 2023, and incorporated by reference in its entirety herein, and illustrates such an augment spreader and delivery device 315 including a deformable plate 321, which may be formed of PEEK or other materials, and first and second retainer mechanisms 7501 which are similar to attachment members 316a, 316b.

As noted above, the portions 312′″ may include two linear strips bounding locations 313a, 313b which create a channel in which attachment members 316a, 316b may engage whereby the portions 312′″ may help hold engagement portions 317a, 317b of the members 316a, 316b in position on augment 310. FIG. 4E shows a section view along plane 319 (shown in FIG. 4B), illustrating the interaction of the attachment member 316a of the augment spreader and delivery device 315 with the portions 312″′ of the augment 310. As shown in FIG. 4E, the attachment member 316a acts as a cantilevered spring arm that biases the engagement portion 317a toward the main body of the augment spreader and delivery device 315 (e.g., downward, as shown). The augment 310 may be inserted into the augment spreader and delivery device 315 from the left, as shown, until the engagement portions 317a, 317b are captured between portions 312′″. Thus, portions 312″′ may act as a detent for the engagement portion 317a. In some embodiments, the engagement portions 317a, 317b and portions 312″′ may be shaped such that the engagement portions 317a, 317b do not contact the first material 311 between the portions 312′″. In other embodiments, the engagement portions 317a, 317b may contact the first material 311 but may still be captured between the portions 312″′ of the second material. In some embodiments, the portion 312′″ may be a single monolithic portion with thicker height portions along each edge and a thinner portion in between, forming a depression where locations 313a, 313b are located. In this variation, engagement portions 317a, 317b of attachment members 316a, 316b would still contact the second material, on portion 312′″, and thus first material 311 would be protected (at least on the surface where portion 312′″ is located) by the second material from the spreader and delivery device. While locations 313a, 313b are illustrated in FIGS. 4A-4B, they need not be physically-defined structures on augment 310. Rather, locations 313a, 313b as illustrated are merely designating the general location where engagement portions 317a, 317b may be positioned relative to portion 312′″.

FIG. 4C provides yet another illustration of a variation of a soft tissue augment, as illustrated augment 310a is largely similar to augment 310 of FIGS. 4A-4B, and thus like reference numbers designate like elements. FIG. 4C illustrates another potential location of a second material portion, such as portions 314, 314′. These exemplary portions 314, 314′, formed as linear strips, may form at least part of a boundary along which attachment members 316a, 316b may be positioned. While the other portions 312, 312′, 312″, 312′″, 312″″ may not need to be included, they are all illustrated in FIG. 4C for purposes of illustrating exemplary potential combinations and variations for positioning second material portions. Thus, as illustrated, attachment member 316a can be bounded by portion 312 and portion 314, while attachment member 316b can be bounded by portion 312′ and portion 314′ (in addition to the aforementioned boundary formed by 312′″). As such, augment 310a demonstrates yet another variation of positioning of the second material portion(s) anywhere on the first material 311 to serve the desired purpose of the user.

FIG. 4D provides yet another illustration of a variation of a soft tissue augment, as illustrated augment 310b is largely similar to augments 310, 310a of FIGS. 4A-4C, and thus like reference numbers designate like elements. FIG. 4D illustrates another potential location of a second material portion, such as portions 318, 318′. These exemplary portions 318, 318′, formed as linear strips, may reinforce a portion of the illustrated augment 310b along which attachment members 316a, 316b may be positioned or may slide across when loading the augment into the device. While the other portions 312, 312′, 312″, 312′″, 312″″ may not need to be included, they are all illustrated in FIG. 4D for purposes of illustrating exemplary potential combinations and variations for positioning second material portions. Thus, as illustrated, when the illustrated augment 310b is inserted into the augment spreader and delivery device 315, attachment member 316a may slide over portion 318 and attachment member 316b may slide over portion 318′, such that the first material 311 is protected by portions 318, 318′. As discussed above, in some embodiments, the engagement portions 317a, 317b and portions 312″′ may be shaped such that the engagement portions 317a, 317b do not contact the first material 311 between portions 312′″. Thus, the engagement portions 317a, 317b may slide across portions 318 and be captured between portions 312″′ without contacting the first material 311, protecting the first material 311 during loading and unloading of the augment 310 into and out of the augment spreader and delivery device 315.

As in the other embodiments, any combination or all of second portions 312, 312′, 312″, 312′″, 312″″, 314, 314′, 318, 318′ illustrated in FIGS. 4A-4D may be monolithic or be one or more separate portions, and indeed, any individual designated portions 312, 312′, 312″, 312′″, 312″″, 314, 314′, 318, 318′ may themselves be multiple portions that are brought together on first material 311 to form the desired pattern. To that end, it should be understood that any lines of demarcation that may be observed within the surface area of the second portions 312, 312′, 312″, 312′″, 312″″, 314, 314′, 318, 318′ or in between one portion and another portion, should not be viewed as limiting, but rather any such lines are simply artifacts from the drafting of the figures. As noted throughout this application, each of the second portions may be any combination of size and shape relative to the other portions or may together be a complete monolithic structure for application to the first material.

As noted above, the various second portions illustrated in the various examples as described above, such as portions 112, 112′, 112″, 112′″ of FIG. 2B, are illustrated as individual rectangular or polygonal second portions or as a monolithic shape having identified sub-portions of the overall shape, but the portions can be any other shapes or sizes or number as desired. For instance, one or more portions could have a shape such as a circle, oval, annulus, triangle, square, or could take on a different shape or size altogether, as either an overall monolithic shape or as individual portions forming a different shape. Still further, fewer portions, or portions of smaller size, could also be applied to the first material 11, or alternatively, additional portions can be added to the first material 11 if desired. In one such example, the end of the first material 11 that is illustrated as free of any second material, could instead have a portion of the second material along that edge (e.g., as in FIG. 3).

Similarly, continuing with the example of portions 112, 112′, 112′, 112′″ of FIG. 2B, while these portions are illustrated as polygonal shapes with defined angular corners, the aforementioned rectangles, triangles, squares, and the like may have rounded or blunt corners; the first material (e.g., material 1 of FIG. 1A) may also have rounded or blunt corners and/or other curved portions (e.g., the lateral edge 1′ could instead be hemispherical or the like). Such curved, rounded, or blunt surfaces may make handling of the soft tissue augment easier as the augment would not have pointed corners that could catch or snag on soft tissue, instruments, sutures, etc. during implantation or once implanted in the patient. Minimizing such snagging may reduce complications during implantation and help minimize irritation of surrounding tissues once implanted in the patient.

FIGS. 5A-5E illustrate some examples of such alternative configurations of the portions of the second material positioned on the first material. As before, each of these second material portions are positioned along any portion of the first material, though the second material portions could be positioned to only partially overlie the first material and at least partially extend beyond an aspect of the outer edge of the first material. FIGS. 5A and 5B illustrate exemplary augments 410, 510 where the portions of second material 412, 412′, 412″, 412′″ and 512, 512′, 512″, 512′″, 512″″, 512′″″, 512″″″ are generally circular in shape and are positioned on first material 411, 511, respectively. As discussed above, certain portions of second material 412, 412′, 512, 512″ may be positioned in the corners of augment 410, 510 (and those corners of first material 411, 511 may also be rounded) for securing the corners of first material 411, 511 to soft tissue, for example, by passing a fixation element (tack, staple, suture, filament, etc.) therethrough and securing the fixation element to native tissue at the implantation site. These positions in the corners may be particularly useful for rotator cuff repair, as these attach the augments 410, 510 at medial locations where ample soft tissue should be present and where ensuring as much contact between soft tissue and augment 410, 510 as possible may be beneficial. Additional second material portions may be positioned elsewhere on first material 411, 511. As illustrated, in the ongoing example of use in rotator cuff repair, these additional second material portions are positioned along the edges of the first material, except for the lateral-most portion of first material 411, 511, though they may be positioned elsewhere on the first material as desired or useful. As shown in FIG. 5B, the augment 510 may additionally include portions of second material 513, 513′ along the edge opposite the portions 512, 512′, 512″, which may correspond to a lateral row in a rotator cuff repair. The portions of second material 513, 513′ may be substantially similar to the portions of second material described above and may reinforce the connection of the fixation element to the augment 510 and provide a more secure attachment of the augment 510 to the native tissue. As shown in FIG. 5D, the augment 710 may additionally include portions of second material 713, 713′, which, like the portions of second material 513, 513′, may reinforce portions of the augment 710 along the edge opposite the portions of second material 714, 714′.

FIGS. 5C and 5D illustrate still further examples of alternative second material portion shape and positioning. In these examples, at least one of the second material portions 612, 612′, 612″ and 712, 712′ are in the shape of a triangle on first material 611, 711, respectively. FIG. 5D includes an example of a combination of shapes, where in addition to triangle second material portions 712, 712′, additional circular second material portions 714, 714′ can also be included.

As with the other examples illustrated and described herein, the first material and the second material portions illustrated in FIGS. 5A-5D could be of any combination of individual or monolithic portions, number of portions, shapes, positioning, sizes, etc. For instance, first material 411, 511, 611, 711 may have any shape desired such as any polygon, rectangle, square, circle, oval, annulus, triangle, or the like. The second portions illustrated in FIGS. 5A-5D could be polygons, circles, triangles, squares, annular, or could take on a different shape or size altogether (both individually and in combination with other portions of second material). Still further, fewer second material portions, or portions of smaller size, could also be applied to the first material, or alternatively, additional portions of the second material can be added to the first material if desired.

In the particular examples of FIGS. 5C and 5D, these combinations of shapes, sizes and positioning may be useful and beneficial for an augment 610, 710 for rotator cuff repair. Commonly in rotator cuff surgery, an augment may be used in combination with a standard “suture bridge” repair, where one or more sutures are positioned through the soft tissue and anchored between two or more suture anchors to secure the soft tissue to bone. An augment may be positioned either on top of or below the soft tissue to assist in support of the soft tissue and potential tissue ingrowth. As illustrated here, the suture bridges 613a, 613b and 713a, 713b are positioned in an “X” shape and the augment 610, 710 is positioned over top of the sutures. Positioning the augment over the suture may be advantageous for the healing process. As discussed throughout this application, where it is desired to minimize the amount of the second material in the augment structure, the second material portions may be shaped and sized to be positioned on the first material 611, 711 but not above the suture bridges 613a, 613b and 713a, 713b because the sewing, felting, stitching, etc. processes to attach the augment to the soft tissue may not be performed in the location of the suture bridges themselves. Thus, the second material portions 612, 612′, 612″ and 712, 712′ may define exposed linear strips of first material that are uncovered by second material above the suture bridges 613a, 613b,713a, 713b. As discussed above, the “linear strips” may not necessarily be rectangular or otherwise include parallel sides, but may allow a line to pass therethrough. For example, as shown in FIG. 5C, the linear strips of exposed first material 611 do not include parallel sides but allow the linear suture bridges to be uncovered by the second material portions 612, 612′, 612″. As such, the second material portions can instead be located elsewhere on the first material 611, 711 to avoid the suture bridges and provide support and material for the sewing, felting, stitching, stapling, anchoring etc. process for attaching the argument to the soft tissue (e.g., directly using tacks, staples, sutures, etc., using suture anchors secured to the soft tissue, etc.). However, in other embodiments, it may be desirable to reinforce the first material above the suture bridges. Thus, in such embodiments, the second material may be arranged to define linear strips on the first material above the suture bridges 613a, 613b,713a, 713b. The linear strips defined by the second material may intersect above the location that the suture bridges 613a, 613b, and 713a, 713b intersect.

FIG. 5E illustrates another augment 760 in accordance with an alternative embodiment. The augment 710 incudes a first layer 761 formed of a first material (as discussed above) and a second layer 762 formed of a second material (as discussed above). The second layer 762 extends around the perimeter of the first layer 761 and also defines linear strips 762′, 762′ that extend across the first layer 761 from corner to corner, intersecting near the center of the first material 761 and defining four triangular portions of exposed first layer 761. In this embodiment, sutures 763a, 763b extend over the augment 760, rather than passing underneath, and are secured to native tissue at the implantation site (e.g., by stitching or by passing through or by securing to anchors that are coupled to the native tissue, such as bone). The linear strips 762′, 762″ that extend across the first material 761 align with and are positioned under the sutures 763a, 763b to protect the first material 761 from abrasion or stress concentrations that may be caused by the sutures 763a, 763b. It should be understood that in other embodiments, the second layer may define linear strips in various shapes and orientations that allow sutures to pass over and secure the augment without contacting the first layer.

With respect to any of the above embodiments and as to the present disclosure overall, the exact placement of the portions of second material relative to the first material may be determined based on the size/shape of the first material and the augment overall, and/or the size and geometry of the augment spreader and delivery device (or other instrument) used to manipulate the augment. According to various aspects, the augment may be configured where the positions of the second material portions are in specific areas where additional strength, support, stiffness, abrasion resistance, etc. are needed. As such, it may be advantageous to balance between minimizing the overall amount of the second material in the augment while having sufficient second material in the appropriate locations to provide adequate strength and support to the augment for manipulation and delivery. In some aspects, less than 100% of the surface area of the first material is covered by the second material (whether with reference to the surface area of the top surface or the bottom surface of the first material), while in other aspects, 100% of at least one surface of the first material may be covered by the second material. According to various aspects, as to the surface of the first material on which the second material is positioned, the second material may cover 70% or less of the total surface area of that surface of the first material. According to other aspects, the second material may cover 60% or less of that surface of the first material, 50% or less of that surface of the first material, 40% or less of that surface of the first material, 30% or less of that surface of the first material, 20% or less of that surface of the first material, 10% or less of that surface of the first material, or any percentage desired (e.g., 15% or less, 25% or less, 33% or less, etc.).

Returning to the exemplary soft tissue augment 100 of FIG. 1B, the portions 2, 2′, 2″ can be attached to the first material 1 as desired. For example, as both first and second materials are likely fibrous materials, a simple sewing or so-called “felting” technique can be used, where a sewing apparatus pushes fibers of the second material forming portions 2, 2', 2″ into and/or through the first material 1. Such a sewing technique can be performed using an apparatus such as the FiberLocker® Instrument SN (Zurimed Technologies AG, Zurich, Switzerland), and may be considered similar to forming a “Velcro®”-like connection between the two layers of material. Such a sewing apparatus may also be used to couple the augment 100 to native soft tissue at an implantation site. For example, the FiberLocker® Instrument SN may be used to push fibers of the second material along portions 2, 2′, 2″ into the native soft tissue at the implantation site, to secure the augment 100 in place. Other attachment techniques may include adhesives, standard stitching (e.g., using a separate yarn or thread to stitch the layers together), or other techniques known in the art. The sewing or felting technique may have advantages over other techniques since it creates many fixation points between the two materials along a surface area which allows for a distributed fixation between the material layers along more of an area than, for instance, if only a couple suturing or stitching points were created in a standard “needle and thread” stitching technique.

In some embodiments, attachment of the portions of second material (such as portions 2, 2′, 2″) to the first material 1 may be performed during manufacture. For example, as discussed above, a complete augment may be manufactured by attaching the second material to the first material using the techniques described herein, including stitching, felting, bonding, adhesives, electrospinning, etc., as well as any other known techniques for attaching such materials. The complete augment, including both the second material attached to the first material, may be provided from the manufacturer to the surgeon. In other embodiments, attaching the second material to the first material can alternatively be performed before or during the actual surgery (e.g., by a member of the surgical staff). For example, first material 1 could be an autograft from the patient themselves, such as a portion of a biceps tendon that can be flattened into a first material 1, and have a square, rectangular, circular, etc. shape. In this example, once the autograft is harvested, the operator in the surgical venue can then attach the graft to the second material using the sewing technique or other techniques noted above. In other examples, any of the techniques for coupling the second material to the first material that may be performed by the manufacturer as discussed above may be performed before or during the surgery (e.g., by a member of the surgical staff), allowing the augment to be customized for the particular surgery, whether an autograft or another first material is used.

In still another example, instead of an autograft, the operator may utilize a standard allograft, such as cadaver allograft material as known in the art. This allograft material typically arrives sterilized, and thus it can be removed from sterile packaging in the operating venue, and then as above, the second material can be attached to the allograft using the sewing technique or other techniques noted above.

In these method examples where the augment 10 is created in the surgical venue, a silicone block or other device might be provided which serves as a base to assist in the attachment technique. The silicone block, as known in the art, may provide a depth below the materials into which a needle, such as from the aforementioned sewing apparatus, would be able to travel to undertake the sewing or felting procedure.

It is envisioned that, particularly for instances where an operator may prepare the soft tissue augment in the operating room, augment materials may be provided in a variety of ways and in a variety of packaging. For example, the second material may be supplied to the user as a variety of pre-formed shapes, such that a user can select the shape(s) and number of second portions desired for a particular application for attachment to the first material. These pre-formed shapes may be trimmed or otherwise further shaped by the user as desired. As such, at least one pre-formed shapes of second material may be supplied to the user as a system or kit, optionally including instrument(s) for attaching the second material portions to the first material, and/or optionally including a first material portion (graft, polymer, or combination, or the like), and/or optionally including a silicone block or other device, all of which may be packaged together or separately as desired. Such packaging may also include instructions for use of the various elements of the system or kit.

In another alternative, the second material portion(s) may be supplied as one or more larger portions, such as a flat sheet, which can then be shaped and divided as desired by the user for subsequent attachment to a first material. As above, this larger portion or sheet of second material may be supplied as a system or kit, and may be packaged together or separately with the other instruments, materials, etc. Such packaging may also include instructions for use of the various elements of the system or kit.

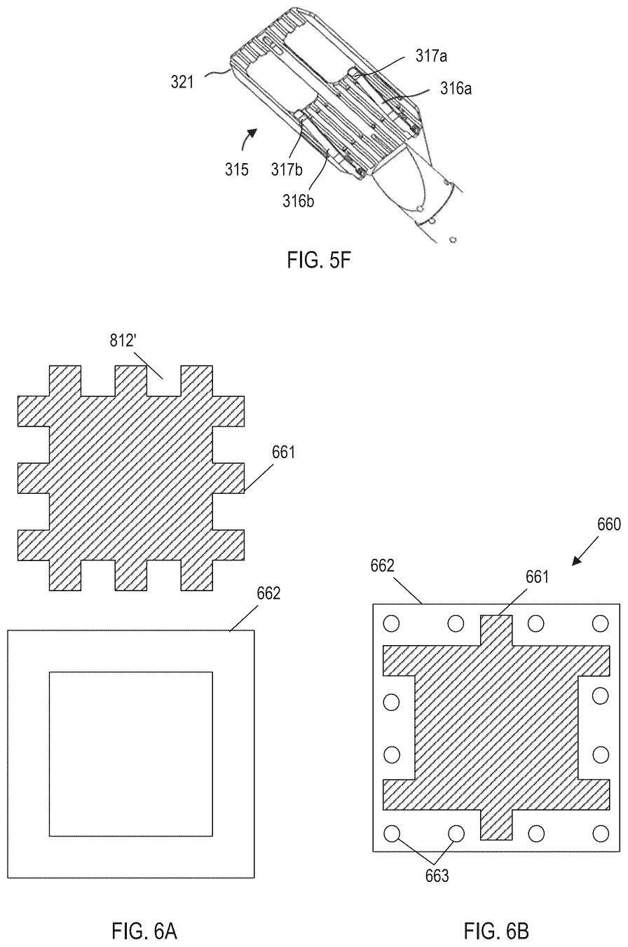

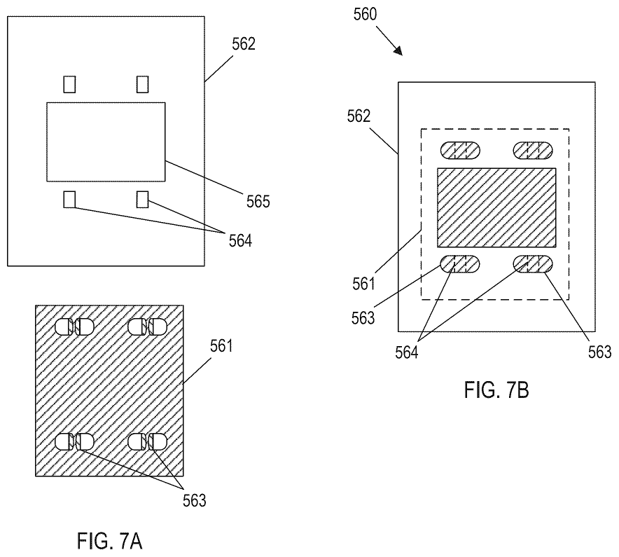

FIGS. 6A-6B and 7A-7B illustrate still further variations on how a first material portion 661 and a second material portion 662 may be combined into a soft tissue augment 660. Looking first to the example of FIG. 6A, the first material portion 661 is illustrated as a puzzle-piece shape while the second material portion 662 is illustrated as a picture-frame shape. FIG. 6B shows one example of how these two portions may be positioned together, where the puzzle-piece extensions of the first material portion 661 may be positioned in an interlocking pattern relative to the picture-frame shaped second material portion 662, whereby the extensions of the first material portion 661 are positioned in an alternating “over-under” interlocking or woven pattern relative to the second material portion 662. In this manner, the first and second material portions 661, 662 need not be attached to one another, as described above, though such an attachment process may be performed (e.g. sewing, felting, stitching, adhering, etc.) if desired, likely at the overlapping junctures of the extensions of the first material and the second material. FIG. 6B illustrates circular locations 663 along the second material portion 662 where the formed augment 610 may be attached to the adjacent soft tissue. Optionally, these locations could be physically marked on the augment. These illustrated locations designate attachment locations where none of the first material is present, though as noted above other attachment locations are also envisioned, including attachment to soft tissue at the locations over overlap between the first and second materials, which as noted above can occur where the second material directly contacts the soft tissue and/or where the first material is positioned in between the second material and the soft tissue. The various other examples discussed above are also applicable here, such as the inclusion of a second piece of first material or second material which may similarly be interwoven with the other materials present, or additional portions of second material may be added onto the surface area of the first material. Still further, the structure could be swapped whereby the second material includes extensions coming off of the “picture-frame” shape which interlock or weave with a generally square or rectangular first material.

FIGS. 7A and 7B illustrate another soft tissue augment 560 with a configuration similar to that of FIGS. 6A-6B, though instead of the interlocking or woven pattern, the first material 561 and second material 562 are instead attached to one another through a “rivet” like configuration. The formed rivets 563 are projections or flaps in the first material 561 formed by laser cutting, standard cutting, forming into the structure during the forming process of the first material (e.g., when molding the polymeric shape of the first material), or the like. As illustrated, these rivet shapes may be positioned through corresponding openings 564 in the second material 562, which are similar formed by cutting or forming techniques, or the like. The projections or flaps of the formed rivets 563 may then be folded downward toward the second material 562, or they may be biased towards such a position, forming hook-like structures that couple the first material 561 to the second material 562. The second material 562 further includes a central opening 565 exposing a larger portion of the first material 561. As with the prior variation, the first and second materials 561, 562 need not be otherwise attached to one another, but any of the aforementioned attachment techniques may once again be used to attach the first and second materials 561, 562 to one another more securely. For example, the outer perimeter of the first material 561 may be stitched to the outer perimeter of the second material 562. Any of the soft tissue attachment techniques and other alternatives noted as to FIGS. 6A-6B are equally applicable here. Still further, the “rivet” shapes may instead be formed on the second material 562 which can engage openings formed on the first material 561, and any other rivet-opening combination formed on the first and second materials 561, 562 envisioned may also be incorporated.

As with the other examples above, the overall shapes of the first and second materials of FIGS. 6A-6B and 7A-7B may be any desired, including the use of rounded corners and other shapes than the illustrated rectangular/square shapes. Similarly, other physical attachments between the first and second materials are also envisioned, such as the formation of multiple warp and weft strips of first/second materials woven together (or as in other variations, the first and/or second materials may represent more than one material each), the configuration where strips of one material weave through a “ladder” shape of the other material, alternating concentric shapes of first and second materials (e.g., which can be secured to one another edge-to-edge), or the like.

As discussed above, in any of the embodiments described herein, the second material layer may include portions of varying thickness. For example, FIGS. 8A-8B illustrate another soft tissue augment 810 with a configuration similar to those of FIGS. 1A-5D. The soft tissue augment 810 includes a first layer 811, which may be formed of a first material (as described above), and a second layer 812, which may be formed of a second material (as described above). The second layer 812 includes first portions 812′ having a first thickness along opposite edges of the first layer 811 and second portions 812″ having a second thickness that is significantly greater than the first thickness along the other two edges of the first layer 811. As used herein “significantly greater” means a difference greater than what would be expected due to standard manufacturing tolerances. Different thicknesses along the various portions of the second layer 812 may allow for customization of stiffness and/or strength of the soft tissue augment 810 along various axes. For example, referring to FIG. 8A, because the second portions 812″ are thicker than the first portions 812′, the soft tissue augment 810 may be stiffer and/or stronger along a horizontal axis and less stiff and/or strong along a vertical axis. Customizing the thicknesses of the portions of the second layer also allows for additional reinforcement material (i.e., second layer material) in areas where the greatest stress, strain, and/or abrasion is expected, including locations in which fixation elements are passed through the augment and locations that interface with various tool, such as the augment spreader and delivery devices described herein. As discussed above, the first portions 812′ and second portions 812″ may be formed of a single piece of material or may be formed of different pieces of material. In some embodiments, for example, a first piece of the second material may be attached to the first layer 811 that surrounds the entire border of the first layer 811, and additional pieces of the second material (or a different material) may be positioned on top of and attached to the first piece of the second material to form portions with greater thickness (e.g., second portions 812″). As discussed above, in any embodiment disclosed herein, the thickness of the second material may vary across the surface of the first material. Further, the embodiments disclosed therein are examples; the second material portions may be positioned in any location on the first material including locations not explicitly shown in the example embodiments. In some embodiments, for example, the entire surface of the first material may be covered by a thin layer of the second material, and certain portions of the augment will have a thicker layer of the second material to further reinforce the augment.

In some embodiments, where a material deposition device (e.g., an electrospinner) is used to form the second layer 812, portions of the first layer 811 and the second layer 812 may be masked to control the placement and thickness of portions of the second layer 812. For example, after depositing a first application of the second material around the entire perimeter of the first layer 811 with a first thickness, the first portions 812′ may be masked while additional material is deposited on the second portions 812″. The mask may then be removed along with any material deposited thereupon, leaving the first portions 812′ at the first thickness and the second portions 812″ at a greater thickness. In other embodiments in which the second material is deposited by electrospinning, material may be deposited without masking. Instead, the electrospinning process can have parameters set which may control the thickness of the second material deposited at each location by the number of passes or the amount of time a head of the electrospinning machine loiters at each location.

FIG. 8C illustrates a soft tissue augment 810′ similar to the soft tissue augment 810′ of FIGS. 8A-8B. However, instead of stepwise transitions between thicknesses of the various portions of the second material 814, the thickness gradually increases from the first thickness of the first portion 814′ to the second thickness of the second portion 814″. This may help to reduce stress concentrations at the interfaces between portions of different thicknesses.

FIGS. 9A-9C illustrate another soft tissue augment 910 with a configuration similar to that of FIGS. 8A-8B, including a first layer 911 and a second layer 912. However, rather than the thicker portions of the second layer 912 extending across entire edges of the first layer 911, the thinner portions 912′ are positioned along each edge, and the thicker portions 912″ are positioned only at the corners. The thicker portions 912″ of soft tissue augment 910 may be positioned at or surrounding attachment points of the soft tissue augment 910 to reinforce the attachment of soft tissue augment 910 to native tissue at the implantation site. For example, as shown in FIG. 9A, the soft tissue augment 910 defines apertures 913 extending through the thicker portions 912′ of soft tissue augment 910. The apertures 913 are each configured to receive a fixation element (e.g., sutures, staples, pins, tacks, etc.) therethrough to secure the soft tissue augment 910 to native tissue at the implantation site. The increased thickness of the second layer 912 surrounding the apertures 913 may reinforce the connection of the soft tissue augment 910 to the native tissue to resist tearing of the soft tissue augment 910.

Although shown in detail in FIG. 9A, it should be understood that any of the embodiments disclosed herein may include features for securing the augment to native tissue at an implantation site. For example, any of the embodiments may include openings extending through the second layer through which filaments, sutures, tacks, staples, or other fixation elements may be passed to secure the augment to the native tissue. The second material layer may reinforce the first material layer to reinforce the openings and resist tearing of the first material when handling and/or securing the augment. The openings may include grommets or eyelets that may reinforce the opening and further protect the augment from tearing. In other embodiments, fixation elements (e.g., tacks, staples, filaments, sutures, etc.) may be passed directly through the second material layer, which may reinforce the fixation location even without a predefined opening. Sutures may be threaded directly to the native tissue or may be threaded through fixation anchors affixed to the native tissue. In some embodiments, the openings or reinforced portions of the augment may be used to guide (e.g., parachute, zipline, etc.) the augment into the implantation site. For example, sutures may be coupled to anchors at the implantation site in a patient and passed through the openings in the augment (e.g., with the augment positioned outside the patient). The augment may then be advanced along the filaments into the patient to the implantation site, and the filaments may be used to secure the augment. Suture shuttles, finger traps, and other known methods of suture coupling may be used to secure the augment to the native tissue.

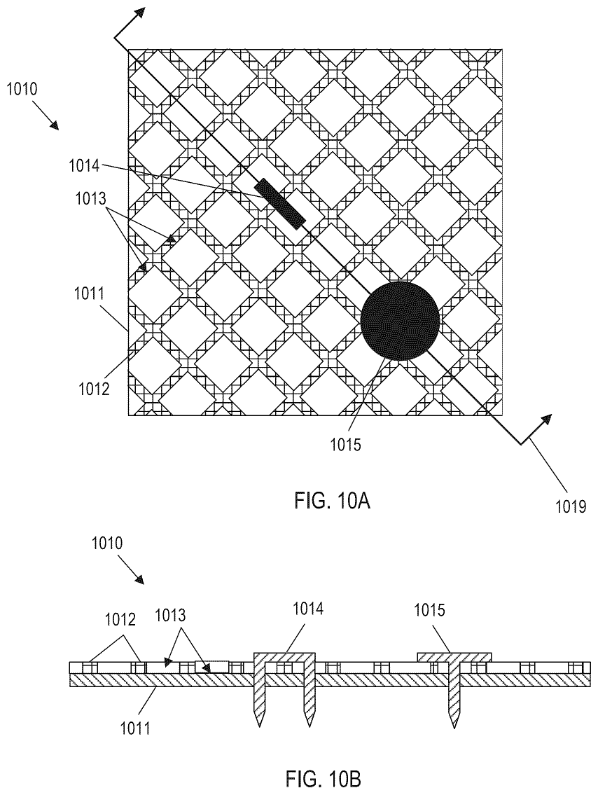

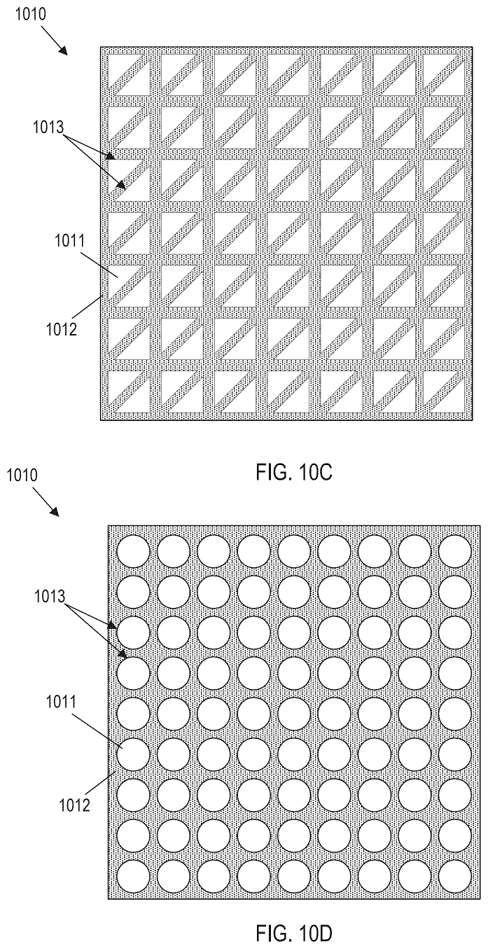

FIGS. 10A-10B illustrate another soft tissue augment 1010 with a configuration similar to those of FIGS. 1A-5D. The soft tissue augment 1010 includes a first layer 1011, which may be formed of a first material (as described above), and a second layer 1012, which may be formed of a second material (as described above). The second layer 1012 is formed in a lattice pattern defining an array of openings 1013 where the first layer 1011 is uncovered. In some embodiments, strips forming a lattice pattern in the second layer 1012 may overlap, while in other embodiments, a lattice-like shape may be formed in a single layer without overlapping strips. In some embodiments, fixation elements (e.g., sutures, staples, pins, tacks, etc.) may pass through the second layer 1012 and the first layer 1011 to secure the soft tissue augment 1010 to native tissue at an implantation site. In other embodiments, and as shown in FIGS. 10A and 10B, the fixation elements, such as exemplary fixation elements 1014, 1015, may pass through only the first layer 1011 and extend over a portion of the second layer 1012. The lattice pattern of the second layer 1012 may resist tearing of the first layer 1011 across the entire soft tissue augment 1010 without fully covering the first layer 1011. FIGS. 10C and 10D show alternative embodiments of the soft tissue augment 1010 each with a second layer 1012 formed in an alternative pattern on the first layer 1011 and defining a plurality of openings 1013 extending therethrough. In some embodiments, spacing between openings 1013 may range from about 1 mm to about 10 mm (center to center) and the lattice “strips” may range from about 1 mm to about 5 mm in width depending on augment size and application. Such lattice configurations may provide a user (e.g., a surgeon) with visual confirmation that the fixation element has passed through the first layer 1011 while the second layer 1012 resists tearing of the first layer 1011.

It should be noted that various patterns other than those shown may be used in different embodiments. For example, while FIGS. 10A and 10C respectively show strips extending in two and three directions, in other embodiments, strips may extend in four or more directions. Additionally, the strips may extend at different angles than as shown and may have different widths than as shown. For example, in some embodiments, the width of each of a first set of strips extending at a first angle may be different than a width of each of a second set of strips extending at a second angle. As another example, the width of each strip extending at a first angle may vary. The spacing between strips may also vary. As shown in FIG. 10D, the pattern may not necessarily be a “lattice” pattern formed of strips but may include a pattern or array of openings through which a fixation element may extend. The openings may vary in shape, size, and spacing in any embodiment. In some embodiments, the pattern of openings and/or strips of second layer 1012 may cover less than the entire surface of the first layer 1011. Portions not covered by the pattern may be uncovered or may be covered by second layer material without openings (e.g., reinforcing strips around the perimeter of the first layer 1011 with the lattice pattern in the center, partially or fully surrounded by the reinforcing strips). In some embodiments, different portions of the second layer 1012 may have different patterns. In some embodiments, the second layer 1012 may cover the entire surface of the first layer 1011 and the openings 1013 may be replaced by a thinner layer of the second layer material. For example, a thin layer of second layer material may be applied to the entire surface of the first layer 1011, and the lattice pattern may be applied on top of the thin layer of second layer material to form a thicker portion of the second layer 1012 in the lattice pattern. Thus, any lattice patterns or patterns of repeating openings (e.g., openings 1013) may be used.

FIG. 10B is a section view along plane 1019 (shown in FIG. 10A), illustrating the interaction of exemplary fixation elements 1014, 1015 and the second layer 1012 of the augment 1010. The first fixation element 1014, shown as a U-shaped staple, includes two legs that extend through adjacent openings 1013 and through the first layer 1011 into the native tissue and a crown or crossbar that extends over a portion of the lattice of second layer 1012. Thus, the first fixation element 1014 captures the lattice between the crossbar and the native tissue without piercing the lattice. The second fixation element 1015, shown as a T-shaped staple or tack includes a single leg that extends from the head of the tack through an opening 1013 in the lattice and through the first layer 1011 into the native tissue. The head of the fixation elements 1015 extends over the lattice of the second layer 1012. Thus, the second fixation element 1015 captures the lattice between the head and the native tissue without piercing the lattice.

FIGS. 11A-11B illustrate another soft tissue augment 1110 that includes a first layer 1111, which may be formed of a first material (as described above), a second layer 1112 on a first surface of the first layer 1111, a third layer 1113 on a second surface of the first layer 1111, and an edge layer 1114 extending around the edge of the first layer 1111 and connecting the second layer 1112 to the third layer 1113. In some embodiments, second layer 1112, third layer 1113, and edge layer 1114 may each be formed of a second material (as described above). In some embodiments, the second layer 1112, third layer 1113, and edge layer 1114 may each be made of different materials. As shown in FIGS. 11A-11B, the second layer 1112 extends across the entire first surface of the first layer 1111 and beyond the edges of the first layer 1111. The third layer 1113 extends around the perimeter region of the second side of the first layer 1111. The second layer 1112, third layer 1113, and edge layer 1114 thus form a pocket or sandwich that captures the first layer 1111 therein, with the first layer 1111 at least partially exposed through the opening or “window” in the third layer 1113. In other embodiments, the first layer 1111 may be fully enclosed on both sides by the second layer 1112 and the third layer 1113. For example, in some embodiments, one or both of the outer layers 1112, 1113 may be made from a biological or degradable synthetic material such that the first layer 1111 is exposed as the outer layer degrades. In some embodiments, the third layer 1113 may not extend around the entire perimeter of the first layer 1111 but rather extends only around a portion or portions of the perimeter. In this way, more of the first layer 1111 may be exposed to the native soft tissue at the implantation site In some embodiments, rather than forming the pocket around the sheet of first material, a pocket with one open end may be formed of the second material, the sheet of first material may be inserted into the pocket, and the open end may be stitched closed (or closed by other means such as with adhesive, etc.). Like the soft tissue augment 1110, such a pocket may include an opening or “window” exposing a portion of the sheet of first material. Still other embodiments may be a reverse of this configuration, with the second material layer positioned in a pocket formed from the first material (e.g., a polyester layer encased in collagen).

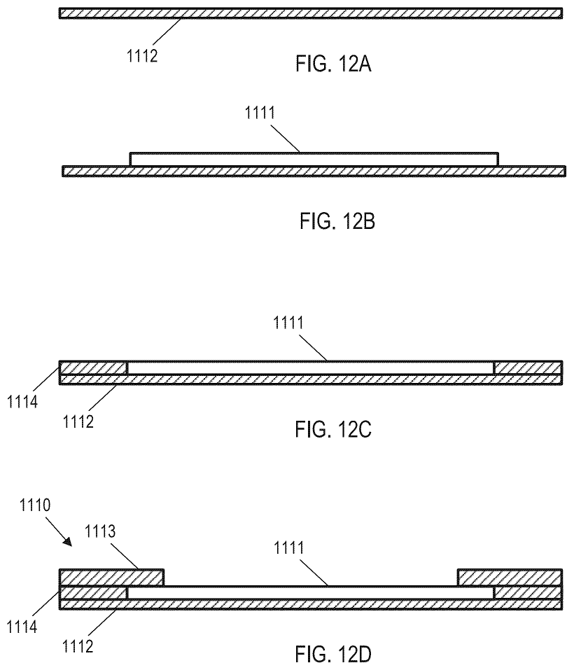

FIGS. 12A-12D illustrate a method of forming the soft tissue augment 1110. As shown in FIG. 12A, the second layer 1112 is fabricated first. The second layer 1112 may be formed in its final shape or cut from a larger piece of material. The second layer 1112 (as well as the third layer 1113 and the edge layer 1114) may be produced as a woven or nonwoven synthetic material or may be formed by spunlacing, electrospinning, or another material deposition process. As shown in FIG. 12B, the first layer 1111 is then laid or formed on top of the second layer 1112. The first layer 1111 may have a surface area smaller than the surface area of the second layer 1112, such that the second layer 1112 extends beyond all of the edges of the first layer 1111. As shown in FIG. 12C, the edge layer 1114 is them formed or positioned on top of the second layer 1112, surrounding the edges of the first layer 1111. The thickness of the edge layer 1114 may be approximately equal to the thickness of the first layer 1111. In some embodiments, where a material deposition process (e.g., electrospinning) is used to form the edge layer 1114, the upper surface of the soft tissue augment 1110 may be masked to ensure that material is not deposited on the upper surface. The mask may then be removed along with any material deposited thereupon.

As shown in FIG. 12D, the third layer 1113 is then formed or positioned on top of the edge layer 1114 and the perimeter portion of the first layer 1111. As with the formation of the edge layer 1114, where a material deposition process is used to form the third layer 1113, a portion of the upper surface of soft tissue augment 1110 may be masked. The mask may be smaller than the mask used to form the edge layer 1114, such that the third layer 1113 is deposited on the perimeter portion of the soft tissue augment 1110. The mask may then be removed with any material deposited thereupon, exposing the center portion of the upper surface of the soft tissue augment 1110.

The various layers 1111, 1112, 1113, 1114 may be coupled together at any of the stages of the assembly process illustrated in FIGS. 12B-12D. As discussed above, the layers 1111, 1112, 1113, 1114 may be coupled together by sewing, felting, adhering, stitching or other methods. In some embodiments, the material of the third layer 1113 and the edge layer 1114 may be applied using electrospinning, and the material may adhere to the first layer 1111 and the second layer 1112 as the material is deposited. In some embodiments, the first layer 1111 may be held in place by the structure of the other layers 1112, 1113, 1114 and may not be otherwise coupled (e.g., may not be stitched, felted, adhered, or sewn) to the other layers 1112, 1113, 1114.

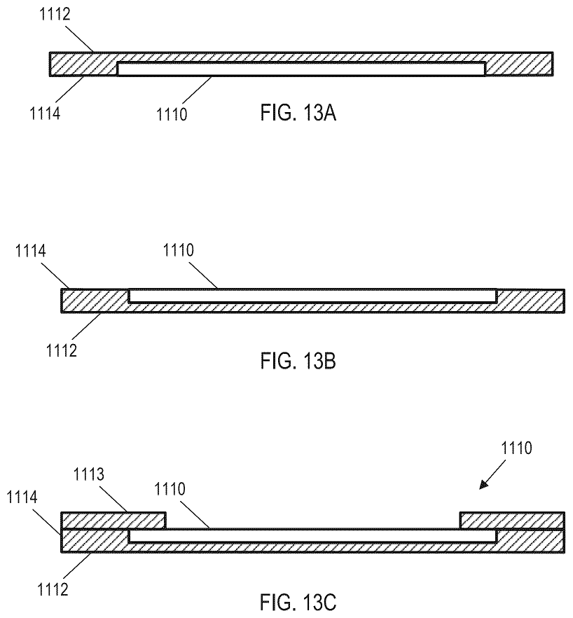

FIGS. 13A-13C illustrate another method of forming the soft tissue augment 1110 using electrospinning or another additive material deposition process. As shown in FIG. 13A, a material deposition device (e.g., an electrospinner) deposits the material of the second layer 1112 and the edge layer 1114 from above onto a first side of the first layer 1111 and around the edges of the first layer 1111. The material of the second layer 1112 and the edge layer 1114 may adhere to the first layer 1111 as it is deposited. As shown in FIG. 13B, the first layer 1111 and the adhered layers 1112, 1114 are then flipped over so that the second side of the first layer 1111 is facing the material deposition device. As shown in FIG. 13C, the material deposition device deposits the material of the third layer 1113 onto the edge layer 1114 and the perimeter region of the second side of the first layer 1111. As discussed above, the material may adhere to the first layer 1111 and the edge layer 1114 as it is deposited.

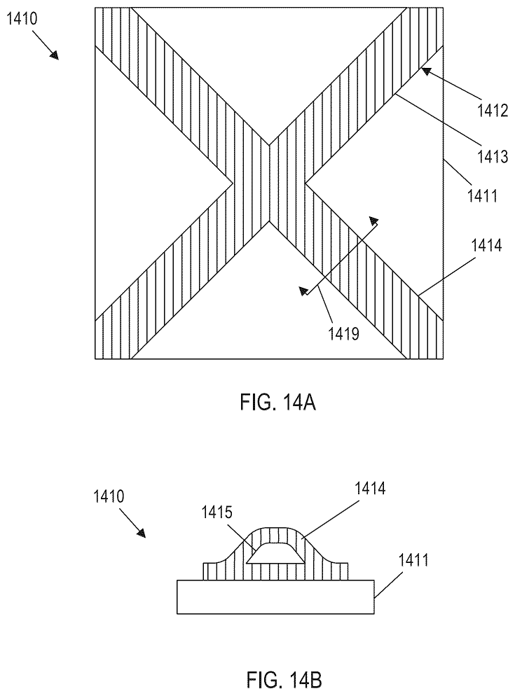

FIGS. 14A-14B illustrate another soft tissue augment 1410 that includes a first layer 1411, which may be formed of a first material (as described above) and a second layer 1412, which may be formed of a second material (as described above). The second layer 1412 includes two portions 1413, 1414 each extending across the first layer 1411 from opposite corners and intersecting at the center of the first layer 1411. As shown in FIG. 14B, a section view along plane 1419 (shown in FIG. 14A), the two portions 1413, 1414 each define an enclosed tunnel 1415 through which a filament or suture may be passed for coupling the soft tissue augment 1410 to native tissue at the implantation site. For example, a suture may be coupled to a first anchor affixed to the implantation site, passed through the tunnel 1415 of the first portion 1413, and coupled to a second anchor affixed to the implantation site. A second suture may be coupled to a third anchor affixed to the implantation site, passed through the tunnel 1416 of the second portion 1414, and coupled to a fourth anchor affixed to the implantation site. It should be understood that this is one of many examples, and that the tunnels 1415, 1416 generally enable the passing of a filament or suture therethrough for coupling the soft tissue augment 1410 to native tissue at the implantation site. For example, tunnels 1415, 1416 may intersect where they meet one another, or one of the tunnels may pass over top of the other tunnel such that they extend past one another, but do not intersect. In other examples, tunnels may extend in any direction along the first material and may or may not intersect or extend past or pass over or under another tunnel. Still further, alternative tunnel shapes may also be used, such as segmented or intermittent tunnels, tunnels that fork or otherwise transition to two or more tunnels, tunnels with different cross-sections, or the like. In some embodiments, one or more tunnels may be used to guide (e.g., parachute, zipline, etc.) the augment into the implantation site. For example, sutures may be coupled to anchors at the implantation site in a patient and passed through the tunnels 6 (e.g., with the augment positioned outside the patient). The augment may then be advanced along the filaments into the patient to the implantation site, and the filaments may be used to secure the augment. Suture shuttles, finger traps, and other known methods of suture coupling may be used to secure the augment to the native tissue.

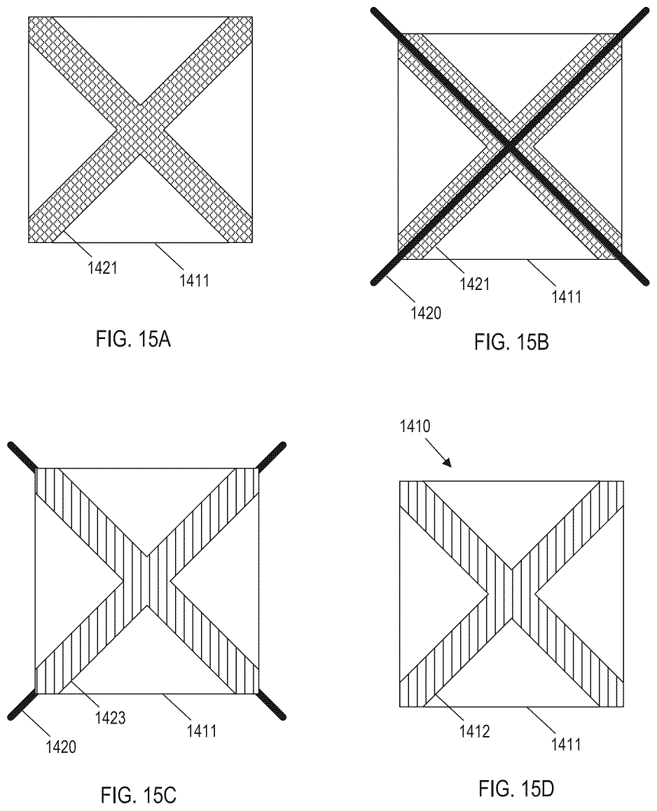

FIGS. 15A-15D illustrate a method of forming the soft tissue augment 1410 using electrospinning or another additive material deposition process. As shown in FIG. 15A, a material deposition device (e.g., an electrospinner) deposits a base layer 1421 of the material of the second layer 1412 onto the first layer 1411, forming the shape of an X, corresponding to the final shape of the two portions 1413, 1414 of the second layer 1412. As shown in FIG. 15B, tubes or rods 1420 are arranged in an X shape and positioned on top of the base layer 1421. As shown in FIG. 15C, the material deposition device deposits another layer 1423 of the material of the second layer 1412 onto the base layer 1421 and the tubes or rods 1420. As shown in FIG. 15D, the tubes or rods 1420 are then removed, leaving behind the second layer 1412 with the tunnels 1415 formed in the locations previously occupied by the tubes or rods 1420. Returning to the augment 100 of FIG. 1B, in one aspect of a method of using augment 100, the augment 100 may be positioned against the soft tissue to which it is to be attached. Augment 100 may be positioned on top of and/or under the soft tissue. The portions 2, 2′, 2″ can be positioned against the soft tissue to which it is to be attached or facing away from the soft tissue. However positioned, augment 100 is then attached to the soft tissue through known techniques, such as sewing or felting, stitching with a separate suture, applying tacks or staples, using adhesive, or the like. In the example of sewing or felting, the operator may use the aforementioned sewing apparatus to push fibers of portions 2, 2′, 2″ into the tissue.

In the example of stitching using a separate suture, the operator may pass the suture through the areas of augment 100 where the portions 2, 2′, 2″ are present. As noted above, attachment is typically performed at least at the corners and also potentially along the edges of the augment 100 to ensure augment 100 lies flat against the soft tissue to establish contact of the soft tissue along as much of the surface area of augment 100, and the respective tissue, as possible.

When portions 2, 2′, 2″ are positioned against the tissue, similar to the manufacturing technique discussed above, a sewing or felting apparatus can be used to push the fibers of portions 2, 2′, 2″ directly into the soft tissue to establish an attachment between augment 100 and the soft tissue. Alternatively, if augment 100 is positioned such that some or all of portions 2, 2′, 2″ are facing away from the soft tissue, the sewing or felting process may provide for the fibers of portions 2, 2′, 2″ to pass through the first material 1 and then into the soft tissue, again creating the attachment between the augment 100 and the soft tissue. While positioning the portions 2, 2′, 2″ directly against the soft tissue may result in better fixation with the soft tissue, the alternative positioning (e.g., with portions 2, 2′, 2″ facing away from the tissue) may result in improved contact between first material 1 and the soft tissue, resulting in an even greater surface area of contact with the augment 100, which may potentially allow for even greater tissue ingrowth.

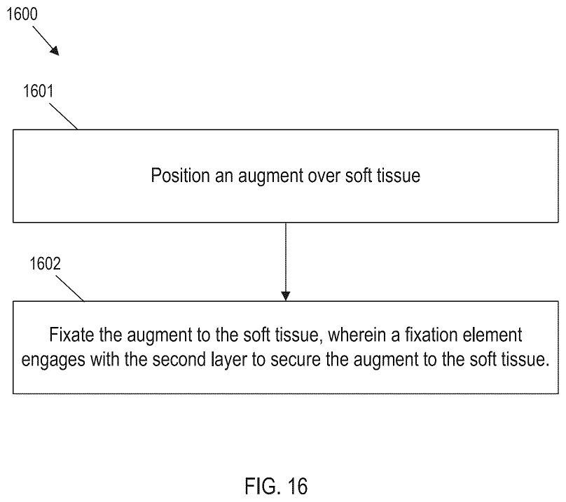

Referring now to FIG. 16, a method 1600 of fixating an augment is shown according to an exemplary embodiment. At operation 1601 of the method 1600, an augment is positioned over soft tissue. The augment may be, for example, any of the augments described herein. The augment includes a first layer formed of a first material, the first material being a biological material, a synthetic material, or a composite material including both a biological material and a synthetic material, the first layer defining a first surface comprising a perimeter region surrounding a center region, and a second layer attached to the first surface of the first layer and formed of a second material. The second material may be a biological material, a synthetic material, or a composite material including both a biological material and a synthetic material and may be different from the first material. In some embodiments, the second layer extends from the perimeter region into the center region.

At operation 1602 of the method 1600, the augment is fixated to the soft tissue, wherein a fixation element engages with the second layer to secure the augment to the soft tissue. In some embodiments, the fixation element pierces through the second layer and into the soft tissue. For example, a tack or staple may be pressed through the second layer and into the soft tissue to secure the augment to the soft tissue as shown in FIG. 1F, or a suture may be passed through the second layer and stitched to the soft tissue or coupled to a suture anchor. In some embodiments, a portion of the fixation element contacts the second layer without piercing through the second layer. For example, a head of a tack or a crown or crossbar or a staple may overlap a portion of the second layer without piercing it, as shown in FIGS. 10A and 10B. In some embodiments, the fixation element extends through an opening predefined in the augment. For example, a tack or staple may be inserted into a predefined opening in the augment, such as the openings shown in FIG. 9A. In some embodiments, the fixation element is a suture, and securing the augment to the soft tissue comprises securing the suture to a suture anchor coupled to the soft tissue. For example, as shown in FIG. 5E, the sutures 763a, 763b engage with the linear strips 762′, 762″ and may be secured to suture anchors coupled to the soft tissue.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A soft tissue augment, comprising:
a first layer formed of a first material, the first material being a biological material, a synthetic material, or a composite material including both a biological material and a synthetic material, the first layer defining a first surface comprising a perimeter region surrounding a center region; and
a second layer attached to the first surface of the first layer and formed of a second material, the second material being a biological material, a synthetic material, or a composite material including both a biological material and a synthetic material, the second material being different from the first material, the second layer extending from the perimeter region into the center region.

2. The soft tissue augment of claim 1, wherein the second material has a greater strength than the first material.

3. The soft tissue augment of claim 1, wherein the second layer covers less than half of the surface area of the first layer.

4. The soft tissue augment of claim 1, wherein the second layer is attached to the first layer by sewing, felting, stitching, bonding, or combinations thereof.

5. The soft tissue augment of claim 1, wherein the second layer is formed of a biological material or a biodegradable polymeric material.

6. The soft tissue augment of claim 1, wherein the second layer extends beyond an outer perimeter defined by the first layer.

7. The soft tissue augment of claim 1, wherein the second layer comprises at least two portions defining a first linear strip of the first surface not covered by the second layer.

8. The soft tissue augment of claim 1, wherein the second layer defines a first linear strip of the second material.

9. The soft tissue augment of claim 8, wherein the second layer further defines a second linear strip of the second material.

10. The soft tissue augment of claim 9, wherein the first linear strip of the second material intersects with the second linear strip of the second material, and wherein the first linear strip and the second linear strip are configured to be positioned under sutures used in a surgical repair.

11. The soft tissue augment of claim 9, wherein the first linear strip and the second linear strip extend parallel across the center region to form a detent for a spring arm of an augment delivery device.

12. The soft tissue augment of claim 1, wherein the second layer is formed in pattern defining a plurality of openings, wherein portions of the first surface aligned with the openings are uncovered by the second layer.

13. The soft tissue augment of claim 1, wherein the second layer comprises a first portion with a first thickness and a second portion with a second thickness greater than the first thickness.

14. The soft tissue augment of claim 13, wherein the second portion with the second thickness is in the perimeter region.

15. The soft tissue augment of claim 1, wherein the first material is a collagen, and the second material is a synthetic material.

16. The soft tissue augment of claim 15, wherein the synthetic material comprises electrospun degradable fibers.

17. The soft tissue augment of claim 1, wherein the second layer defines an opening or a tunnel configured to receive a suture or filament.

18. A method of forming a soft tissue augment, the method comprising:
providing a first layer formed of a first material, the first material comprising a biological material, a synthetic material, or a composite material including both a biological material and a synthetic material, the first layer defining a first surface comprising a perimeter region surrounding a center region; and
coupling a second layer to the first surface of the first layer with the second layer extending from the perimeter region into the center region, the second layer formed of a second material, the second material being a biological material, a synthetic material, or a composite material including both a biological material and a synthetic material, the second material being different from the first material.

19. The method of claim 18, wherein the second material is a nonwoven polymeric material, and wherein coupling the second layer to the first surface of the first layer comprises pressing fibers from the second layer through the first layer.

20. The method of claim 18, wherein coupling the second layer to the first layer comprises sewing, felting, stitching, bonding, or combinations thereof.

21. The method of claim 18, wherein coupling the second layer to the first surface of the first layer comprises depositing the second material using electrospinning.

22. The method of claim 21, wherein the second material is deposited with varying thickness across the first surface of the first layer.

23. The method of claim 21, wherein the second material is degradable.

24. The method of claim 21, wherein the second material is deposited in a first portion with a first thickness and a second portion with a second thickness greater than the first thickness, and wherein at least some of the second portion with the second thickness is located in the perimeter region.

25. The method of claim 18, further comprising positioning the first layer between the second layer and a third layer, wherein coupling the second layer to the first surface of the first layer comprises coupling the second layer to the third layer to capture the first layer between the second layer and the third layer.

26. A method comprising:
positioning an augment over soft tissue, the augment comprising a first layer formed of a first material, the first material being a biological material, a synthetic material, or a composite material including both a biological material and a synthetic material, the first layer defining a first surface comprising a perimeter region surrounding a center region, and a second layer attached to the first surface of the first layer and formed of a second material, the second material being a biological material, a synthetic material, or a composite material including both a biological material and a synthetic material, the second material being different from the first material, the second layer extending from the perimeter region into the center region; and
fixating the augment to the soft tissue, wherein a fixation element engages with the second layer to secure the augment to the soft tissue.

27. The method of claim 26, wherein the fixation element pierces through the second layer and into the soft tissue.

28. The method of claim 27, wherein a portion of the fixation element contacts the second layer without piercing through the second layer.

29. The method of claim 26, the fixation element extends through a predefined opening of the second layer.

30. The method of claim 28, wherein the fixation element is a suture, wherein securing the augment to the soft tissue comprises securing the suture to a suture anchor that is coupled to the soft tissue or to bone.
